(19) United States
(12) Patent Application Publication
(10) Pub. No.: US 2023/0167445 A1
(43) Pub. Date: Jun. 1, 2023

(54) COMPOSITIONS COMPRISING EXOGENOUS HOMOLOGOUS DNA AND USES THEREOF

(71) Applicant: SUPERBREWED FOOD, INC., New Castle, DE (US)

(72) Inventors: Bryan P. TRACY, Wilmington, DE (US); Biniam MARU, Bear, DE (US); Alon KARPOL, Tel Mond (IL); Aharon M. EYAL, Jerusalem (IL)

(21) Appl. No.: 17/922,365

(22) PCT Filed: May 4, 2021
(86) PCT No.: PCT/IB2021/053750

§ 371 Date: Oct. 30, 2022

Related U.S. Application Data
(60) Provisional application No. 63/020,049, filed on May 5, 2020.

(51) Int. Cl. C12N 15/113; A61P 31/04

(52) U.S. Cl. CPC C12N 15/113; A61P 31/04; C12N 2310/111

(57) ABSTRACT

Provided is a composition comprising at least one selected fragment of DNA exogenous to a targeted organism and a carrier, wherein said DNA fragment has at least 50% sequence homology to a DNA sequence of said targeted organism and wherein said fragment comprises no more than 5 unprotected specific endonuclease recognition sites of said targeted organism. Further provided are methods of use of the composition. Further provided are methods for producing the composition.

### CROSS-REFERENCE TO RELATED APPLICATION

The present application gains priority from U.S. Provisional Application No. 63/020,049 filed May 5, 2020, which is incorporated by reference as if fully set-forth herein.

### BRIEF DESCRIPTION OF THE DESCRIBED SEQUENCES

The nucleic acid sequences provided herewith are shown using standard letter abbreviations for nucleotide bases as defined in 37 C.F.R. 1.822. Only one strand of each nucleic acid sequence is shown, but the complementary strand is understood as included by any reference to the displayed strand. The Sequence Listing is submitted as an ASCII text file named WDL34-PC seq listing_ST25, created May 4 2021, about 3 KB, which is incorporated by reference herein.

### FIELD OF THE INVENTION

The present invention, in at least some embodiments, relates to compositions comprising fragments of DNA which are exogenous and homologous to those of a targeted organism, methods of production thereof and uses thereof.

### BACKGROUND OF THE INVENTION

It is known in the art that growth and survival of bacteria are inhibited by autologous DNA. It is further known that such inhibition has not previously been found with exogenous DNA.

### SUMMARY OF THE INVENTION

According to an aspect of some embodiments of the present invention, there is provided a composition comprising at least one selected fragment of DNA exogenous to a targeted organism and a carrier, wherein said DNA fragment has at least 50% sequence homology to a DNA sequence of said targeted organism and wherein said fragment comprises no more than 5 unprotected specific endonuclease recognition sites of said targeted organism.

According to a further aspect of some embodiments of the present invention, there is provided a method for the production of the composition as disclosed herein, comprising (i) providing a DNA fragment having at least 50% sequence homology to a sequence of said targeted organism; (ii) modifying at least one specific endonuclease recognition site of said DNA fragment to provide a modified DNA fragment comprising no more than 5 unprotected specific endonuclease recognition sites of said targeted organism; and (iii) isolating said modified DNA fragment.

According to a further aspect of some embodiments of the present invention, there is provided a method for the treatment of a condition caused by a pathogenic, parasitic or invasive microorganism or a symptom thereof, the method comprising administering to a subject in need thereof a therapeutically effective dose of the composition as disclosed herein.

According to a further aspect of some embodiments of the present invention, there is provided a composition as disclosed herein for use in treating a condition caused by a pathogenic, parasitic or invasive microorganism or a symptom thereof.

According to a further aspect of some embodiments of the present invention, there is provided a method for reducing the use of antibiotics for the treatment of a condition caused by a pathogenic, parasitic or invasive microorganism or a symptom thereof comprising administering a therapeutically effective dose of the composition as disclosed herein.

According to a further aspect of some embodiments of the present invention there is provided a method for at least one of reducing industrial contaminants, reducing biofilm formation, reducing water contamination and bioterrorism defense, the method comprising use of the composition as disclosed herein.

### BRIEF DESCRIPTION OF THE FIGURES

Some embodiments of the invention are described herein with reference to the accompanying figures. The description, together with the figures, makes apparent to a person having ordinary skill in the art how some embodiments of the invention may be practiced.

In the Figures:

FIG. 1 is a bar graph showing inhibition of growth of various strains of microorganisms by genomic DNA from S. aureus.

### DETAILED DESCRIPTION OF THE INVENTION

The present invention, in at least some embodiments thereof, relates to compositions comprising fragments of DNA, which are exogenous and homologous to that of a targeted organism, methods of production thereof and uses thereof.

The present inventors have surprisingly found that exogenous DNA can be used to kill or inhibit growth of a targeted organism, such as a pathogenic microorganism. It was found that autologous DNA from Clostridium tyrobutyricum (Cty) species 1460 (Cty1460) inhibits growth of the microorganism, while DNA from Clostridium butyricum (Cbu) DNA did not inhibit growth of Cty1460, even though it has a high degree of homology. Similar results have been observed in many other strains of Clostridium and related organisms.

Without wishing to be bound by any one theory, the present inventors have hypothesized that inhibition of bacterial growth upon exposure to exogenous DNA having specific characteristics is caused by interference with DNA replication due to binding of single-stranded DNA of the targeted microorganism, which inhibits replication leading to death or inhibition of growth if the microorganism.

It is further hypothesized that lagging strands of synthesized DNA are most vulnerable, which enables preparation of exogenous DNA libraries in plasmids with rolling circles of replication.

It is known that essentially all bacteria have restriction endonuclease systems, which are enzymes that cut double stranded DNA at very specific recognition sequences. Organisms have developed these enzymes to protect themselves against attack by exogenous DNA. Furthermore, cells express exonucleases, which are enzymes that completely degrade DNA by chewing it up from the free ends of DNA. Since bacterial chromosomes occur in the form of a loop, there are no free ends available for cleavage by exonuclease. Upon endonuclease recognition of exogenous DNA, cutting by the enzyme occurs. Each cut leaves two free ends, which the exonucleases then cleave until the DNA is completely degraded.

Bacteria are protected against self-degradation by endonucleases due to the action of methyltransferase enzyme which can modify DNA by methylation, and thereby protect nucleotides at specific recognition sequences. Methylated DNA is protected from restriction endonucleases.

It has thus surprisingly been found that exogenous DNA having a sufficient degree of homology to DNA of a targeted microorganism and which is protected, such as by methylation, is not degraded by restriction endonucleases of the targeted microorganism.

The present inventors further hypothesize that an effective dose of a composition comprising methylated exogenous DNA which would be required for treatment of a condition caused by a targeted microorganism would be ½ to 1/10 of a dose of an antibiotic required to provide the same effect.

Advantages of use of compositions of the present invention as compared to use of antibiotics include an absence of off-target effects; potential use in personalized medical therapy, such as against antibiotic-resistant bacteria; and absence of acquired resistance of bacteria.

An advantage of the use of the compositions as disclosed herein as compared to use of autologous DNA is that use of autologous DNA in treatment of a condition or symptom caused by pathogenic bacteria requires that a large volume of the pathogenic bacteria must be grown in order to isolate sufficient DNA, wherein a much smaller volume of the bacteria is required according to the present invention.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention.

As used herein, the term “selected fragment” means non-random fragments.

As used herein, the term “exogenous DNA” with regard to a targeted species refers to DNA which is not produced naturally by that species. In some embodiments, the exogenous DNA is produced by a different species. In some embodiments, the exogenous DNA is synthesized.

As used herein, the term “sequence homology” with regard to fragments of exogenous DNA refers to the degree of similarity between a nucleotide sequence of at least 20 base pairs in the fragments of exogenous DNA and a nucleotide sequence in the DNA of a targeted species, expressed as a percentage, wherein sequence having 100% homology are identical.

As used herein, the term “targeted organism” refers to an organism, such as a microorganism, which the compositions of the present invention are intended to kill or of which the compositions are intended to at least partially inhibit growth.

As used herein, the term “unprotected” with regard to a recognition site for an enzyme refers to a site which can be cleaved by that enzyme in its unmodified state.

As used herein, the term “recognition site” with regard to an endonuclease enzyme is a nucleotide sequence that is specifically recognized by the enzyme.

As used herein, the term “mutation” refers to an alteration of a nucleotide sequence of a gene. The alteration may comprise a deletion or replacement of a single nucleotide, an insertion (to generate frame-shift, or addition of a stop codon), or a missense (nonsense) mutation. Deletion can be achieved by mutation in the gene itself or in its regulation sites (such as the promoter, ribozyme binding site, repressor binding site, or termination site).

As used herein, the term “treating” includes preventing, curing, ameliorating, mitigating, and reducing the instances or severity of a condition or a symptom thereof.

As used herein, the term “administering” includes any mode of administration, such as oral, subcutaneous, sublingual, transmucosal, parenteral, intravenous, intra-arterial, buccal, sublingual, topical, vaginal, rectal, ophthalmic, otic, nasal, inhaled, intramuscular, intraosseous, intrathecal, and transdermal, or combinations thereof. “Administering” can also include providing a different compound that when ingested or delivered as above will necessarily transform into the compound that is desired to be administered, this type of “different compound” is often being referred to as a “Prodrug”. “Administering” can also include prescribing or filling a prescription for a dosage form comprising a particular compound. “Administering” can also include providing directions to carry out a method involving a particular compound or a dosage form comprising the compound or compounds.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, when a numerical value is preceded by the term “about”, the term “about” is intended to indicate +/−10% of that value.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

According to an aspect of some embodiments of the present invention, there is provided a composition comprising at least one selected fragment of DNA exogenous to a targeted organism and a carrier, wherein said DNA fragment has at least 50% sequence homology to a DNA sequence of said targeted organism and wherein said fragment comprises no more than 5 unprotected specific endonuclease recognition sites of said targeted organism.

According to an embodiment, said DNA fragment has 5, 4, 3, 2, 1 or 0 unprotected specific endonuclease recognition sites.

According to an embodiment, said fragment comprises no more than 2 unprotected specific endonuclease recognition sites of said targeted organism per 1.5 kilobase pairs (kb) of said DNA fragment. In some such embodiments said fragment comprises 2, 1 or 0 unprotected specific endonuclease recognition sites of said targeted organism per 1.5 kb of said DNA fragment.

According to an embodiment, said targeted organism comprises a microorganism selected from the group consisting of Enterococccus, Clostridium, Escherichia, Klebsiella, Campylobacter, Peptococcus, Heliobacter, Hemophylus, Staphylococcus, Yersinia, Vibrio, Shigella, Salmonella, Streptococcus, Proteus, Pseudomonas and Toxoplasmosis and combinations thereof.

According to an embodiment, said at least one DNA fragment has at least 50% sequence homology to said DNA sequence of said targeted organism, such as at least 50%, at least 55%, at least 60%, at least 65%, at least 70%, at least 75%, at least 80%, at least 90%, at least 95% or about 100% homology. In a preferred embodiment, homology is at least 80%.

According to at an embodiment, the number of unprotected specific endonuclease recognition sites is reduced to no more than 5 by protection of the recognition sites, e.g. by chemical or enzymatic modification, mutation or deletion of nucleotides. In some embodiments, chemical modification comprises methylation.

According to an embodiment, said at least one DNA fragment is a synthetic fragment.

According to an embodiment, said synthetic fragment has even number of single stranded DNA overhangs at a 5′ and 3′ end.

According to an embodiment, said at least one DNA fragment comprises an average of at least one methylated endonuclease recognition site of said targeted organism per 1.5 kb of said DNA fragment.

According to an embodiment, said at least one DNA fragment comprise an average of at least one endonuclease recognition site of an organism other than said targeted organism.

According to an embodiment, said at least one fragment comprise between 50 and 50,000 base pairs, such as at least 50, at least 100, at least 200, at least 500, at least 1000, at least 2000, at least 5,000, at least 10,000, at least 15,000, at least 20,000, at least 25,000, at least 30,000, at least 35,000, at least 40,000, at least 45,000 or at least 50,000 base pairs.

According to an embodiment, said carrier is an aqueous solution.

According to an embodiment, said carrier further comprising at least one selected from the group consisting of divalent cations, monovalent cations, dimethyl sulfoxide, dithiothreitol, and hexamine cobalt and combinations thereof.

According to an embodiment, said cations are selected from the group consisting of Mg²⁺, Mn²⁺, Ca²⁺, Rb⁺, K⁺ and combinations thereof.

According to an embodiment, said targeted organism is selected from the group consisting of Enterococccus, Clostridium, Escherichia, Klebsiella, Campylobacter, Peptococcus, Heliobacter, Hemophylus, Staphylococcus, Yersinia, Vibrio, Shigella, Salmonella, Streptococcus, Proteus, Pseudomonas and Toxoplasmosis.

According to an embodiment, said targeted organism is selected from the group consisting of Salmonella enterica and said endonuclease recognition site is GATCAG; Salmonella enterica and said endonuclease recognition site is CAGAG; Salmonella enterica and said endonuclease recognition site is GAGNNNNNNRTAYG (SEQ ID NO:1); Salmonella enterica and said endonuclease recognition site is GGTANNNNNRTGAA (SEQ ID NO:2); Clostridium difficile and said endonuclease recognition site is CANNNNNNNTAAAG (SEQ ID NO:3); Clostridium difficile and said endonuclease recognition site is AYGNNNNNNCTG (SEQ ID NO:4); Clostridium difficile and said endonuclease recognition site is TAGNNNNNRTGAA (SEQ ID NO:5); Staphylococcus aureus and said endonuclease recognition site is AGGNNNNNGAT (SEQ ID NO:6); Staphylococcus aureus and said endonuclease recognition site is SCNGS; Streptococcus pneumoniae and said endonuclease recognition site is TCTAGA; wherein said targeted organism is Streptococcus pneumoniae and said endonuclease recognition site is GATC; Neisseria gonorrhoeae and said endonuclease recognition site is GGCC; Neisseria gonorrhoeae and said endonuclease recognition site is GGNNCC; Neisseria gonorrhoeae wherein said endonuclease recognition site is GGTGA; Neisseria gonorrhoeae and said endonuclease recognition site is GCCGGC; Neisseria gonorrhoeae and said endonuclease recognition site is RGCGCY; Neisseria gonorrhoeae and said endonuclease recognition site is CCACC; Neisseria gonorrhoeae and said endonuclease recognition site is CCGCGG; Neisseria gonorrhoeae and said endonuclease recognition site is GATC; Neisseria gonorrhoeae and said endonuclease recognition site is GCSGC; Neisseria gonorrhoeae and said endonuclease recognition site is GACNNNNNTGA (SEQ ID NO:7); Neisseria gonorrhoeae and said endonuclease recognition site is GAGNNNNNTAC (SEQ ID NO:8); Neisseria gonorrhoeae and said endonuclease recognition site is GCAGA; Campylobacterjejuni and said endonuclease recognition site is CATG; and Campylobacterjejuni and said endonuclease recognition site is GCANNNNNRTTA (SEQ ID NO:9).

According to a further aspect of some embodiments of the present invention, there is provided a method for the production of the composition as disclosed herein, comprising (i) providing a DNA fragment having at least 50% sequence homology to a sequence of said targeted organism; (ii) modifying at least one specific endonuclease recognition site of said DNA fragment to provide a modification wherein said fragment comprises no more than 5 unprotected specific endonuclease recognition sites of said targeted organism; and (iii) isolating said modified DNA fragment.

According to an embodiment, said modification comprises chemical or enzymatic modification.

According to an embodiment, said modification comprises substitution of at least one nucleotide.

According to an embodiment, said modification comprises deletion of at least one nucleotide.

According to an embodiment, said modification comprises methylation.

According to an embodiment, said modifying comprises contacting said fragment with a methyl transferase enzyme.

According to an embodiment, said DNA fragment is double stranded.

According to an embodiment, said DNA fragment is single stranded.

According to an embodiment, said DNA fragment is obtained from a non-pathogenic organism.

According to an embodiment, said DNA fragment is a synthetic sequence.

According to an embodiment, said methylation is carried out in a host cell.

According to an embodiment, said methylation is carried out in vitro.

According to a further aspect of some embodiments of the present invention, there is provided a method for the treatment of a condition caused by a pathogenic, parasitic or invasive microorganism or a symptom thereof, the method comprising administering to a subject in need thereof a therapeutically effective dose of the composition as disclosed herein.

According to a further aspect of some embodiments of the present invention, there is provided a composition as disclosed herein for use in treating a condition caused by a pathogenic, parasitic or invasive microorganism or a symptom thereof.

According to an embodiment, said condition is selected from the group consisting of Crohn's disease, a C. difficile infection, a urinary tract infection, a vaginal infection, a sexually transmitted disease, sepsis, inflammation and combinations thereof.

According to an embodiment, said therapeutically effective dose is from about 0.05 to about pmoles/g cells.

According to an embodiment, said treatment comprises killing said pathogenic, parasitic or invasive microorganism.

A therapeutically effective amount of the composition to be administered is that which, when administered to a subject for treating a condition or a symptom thereof, is sufficient to have a beneficial effect with respect to that condition or symptom thereof. The amount to be administered will vary depending on the specific targeted organism, the specific condition or symptom thereof and its severity, and the age, weight, and other relevant characteristics of the subject to be treated. Determining the therapeutically effective amount of the composition to be administered is within the ordinary skill of the art and typically requires no more than routine experimentation.

According to an embodiment, the composition as disclosed herein is administered at least twice.

According to an embodiment, the composition is administered once a day, twice a day, three times a day or more for a given period.

According to an embodiment, administering once a day comprises administering in the morning. According to an embodiment, administering once a day comprises administering in the afternoon. According to an embodiment, administering once a day comprises administering in the evening.

According to an embodiment, administering twice a day comprises administering in the morning and the afternoon. According to an embodiment, administering twice a day comprises administering in the morning and the evening. According to an embodiment, administering twice a day comprises administering in the afternoon and the evening.

According to an embodiment, administering three times a day comprises administering in the morning, afternoon and evening.

According to an embodiment, administering is carried out once, twice, three times or more per day for one day, two days, three days, four days, five days, six days, one week, two weeks, three weeks, one month, two months, three months, four months, five months, six months, seven months, eight months, nine months, ten months, eleven months, one year or more than one year.

According to an embodiment, administering is carried out by a route selected from the group consisting of enteral (such as oral, sublingual, buccal, gastric, duodenal or rectal) or parenteral (such as IV, intramuscular subcutaneous and transdermal routes).

According to an embodiment, administering is carried out by oral administration. According to some such embodiments, the composition is provided in the form of a solid oral dosage form, such as a tablet, capsule, sachet, powder, granule. According to some such embodiments, the composition is provided in the form of a liquid oral dosage form, such as a syrup, solution or dispersion. According to some embodiments, the composition further comprises one or more excipients, such as sweeteners, colorants, flavorants, stabilizers and the like.

According to an aspect of some embodiments of the present invention, there is provided a method for reducing the use of antibiotics for the treatment of a condition caused by a pathogenic, parasitic or invasive microorganism or a symptom thereof comprising administering a therapeutically effective dose of the composition as disclosed herein.

According to a further aspect of some embodiments of the present invention there is provided a method for at least one of reducing industrial contaminants, reducing biofilm formation, reducing water contamination and bioterrorism defense, the method comprising use of a composition as disclosed herein. According to some such embodiments, the composition is provided in a liquid form.

### EXAMPLES

Materials and Methods

i. Preparation of Bacteria

A single bacterial colony of age no greater than 4 days was selected from an agar plate and inoculated into 5 ml of medium (according to Table 1 below) in a 10 ml test tube. The culture was grown overnight at a temperature according to Table 1 below.
The following morning, 100-250 μl of the overnight culture was inoculated into fresh medium in a second 10 ml test tube to provide a starter culture. The starter culture was allowed to grow until mid-log phase was reached.

TABLE 1
Growth media and growth conditions

| | | | OD mid- |
|---|---|---|---|
| Bacteria | Medium | Temperature | log¹ |
| Staphylococcus aureus | CGM | 37° C. | 0.2-1.0 |
| Staphylococcus epidermidis | CGM | 37° C. | 0.2-0.8 |
| E. coli | CGM | 37° C. | 0.2-0.8 |
| C. difficile | CGM | 37° C. | 0.2-0.8 |

ii. Inhibition Assay

A DNA master stock was provided having a concentration of no greater than 2,000 μg/ml.

Cells of the starter culture in mid-log phase were diluted to OD of 0.05 with growth medium.

Serial dilution samples of the DNA master stock were prepared in test tubes according to Table 2 below.

The assay included the following controls:

a) Control 1 (tube no. 8): DNA and medium, no cell culture (to check for contamination of DNA);

b) Control 2 (tube no. 9): Medium alone, no cell culture, no DNA (to check for contamination of medium).

TABLE 2
Sample preparation (Inoculation 50% volume)

| Tube no. | | 1 | 2 | 3 | 4 | 5 | 6 | 7 | | |
|---|---|---|---|---|---|---|---|---|---|---|
| (μg/ml) | | 400 | 200 | 100 | 50 | 25 | 12.5 | 0 | 8 | 9 |
| Substrate | | Stock | | | | | | | | |
| DNA | 2000 | 200 | 100 | 50 | 25 | 12.5 | 6.25 | 0 | 50 | 0 |
| | ng/ul | | | | | | | | | |
| DD Water | | 0 | 100 | 150 | 175 | 187.5 | 193.75 | 200 | 50 | 0 |
| Medium | | 300 | 300 | 300 | 300 | 300 | 300 | 300 | 900 | 1000 |
| Starter | 0.05 | 500 | 500 | 500 | 500 | 500 | 500 | 500 | 0 | 0 |
| Culture | OD | | | | | | | | | |

iii. Measurement of Growth

The tubes were inspected visually on a daily basis for growth of cells, using the control samples as reference. Once growth was observed in tubes 5 and 7 having no DNA content, a 200 μl aliquot was withdrawn from each of tubes 1-7 and absorbance measured at 595 nm, using 96-well plates.

The concentration of DNA which provided 50% inhibition of growth (IC50) was calculated.

### Example 1: Growth Inhibition by Genomic DNA from Staphylococcus aureus

Genomic DNA was extracted from Staphylococcus aureus by phenol: chloroform extraction and purified using a PD-10 desalting column comprising Sephadex G-25 resin.

Cultures of the following strains were prepared as described above and each strain was inoculated to a suitable growth medium as detailed in Table 1:

a. Staphylococcus aureus

b. Staphylococcus epidermidis

c. Escherichia coli

d. Cutibacterium acne).

Purified DNA was added to each culture at one of the following concentrations: 0, 5, 25, 50, 100, 200 and 400 ug/ml.

Cell growth was determined by optical density (OD) measurements at 600 nm.

A linear plot of OD versus DNA concentration was prepared, and the value DNA concentration at which 50% growth inhibition was seen (ID50) was determined.

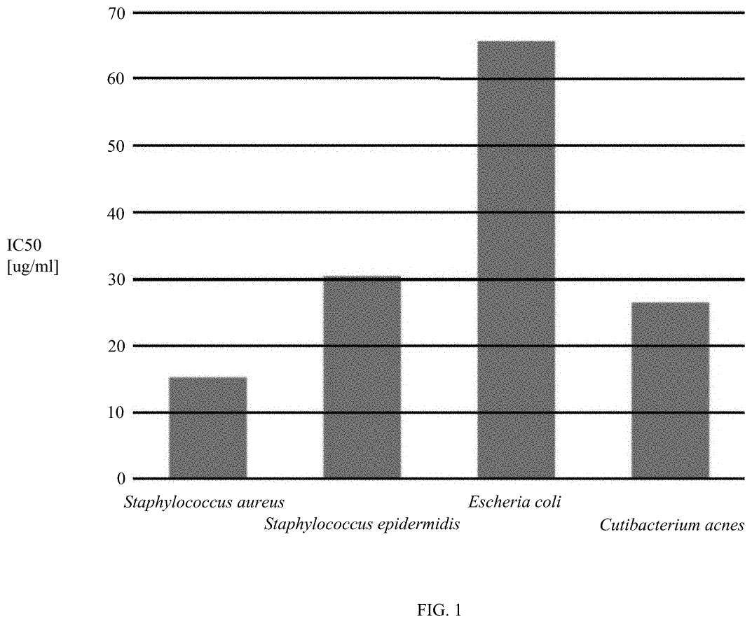

Results are presented in FIG. 1, showing that the highest inhibition (represented by the lowest concentration of DNA required to provide 50% inhibition) was seen by DNA from S. aureus against itself, while lowest inhibition was seen against E. coli.

1. A composition comprising at least one selected fragment of DNA exogenous to a targeted organism and a carrier, wherein said DNA fragment has at least 50% sequence homology to a DNA sequence of said targeted organism and wherein said fragment comprises no more than 5 unprotected specific endonuclease recognition sites of said targeted organism.

2. The composition of claim 1, wherein said fragment comprises no more than 2 unprotected specific endonuclease recognition sites of said targeted organism per 1.5 kilobase pairs of said DNA fragment.

3. The composition of claim 1, wherein said targeted organism comprises a microorganism selected from the group consisting of Enterococccus, Clostridium, Escherichia, Klebsiella, Campylobacter, Peptococcus, Heliobacter, Hemophylus, Staphylococcus, Yersinia, Vibrio, Shigella, Salmonella, Streptococcus, Proteus, Pseudomonas and Toxoplasmosis.

4. The composition of claim 1, wherein said at least one DNA fragment has at least 80% sequence homology to said DNA sequence of said targeted organism.

5. The composition of claim 1, wherein said at least one DNA fragment is a synthetic fragment.

6. The composition of claim 1, wherein said synthetic fragment has even number of single stranded DNA overhangs at a 5′ and 3′ end.

7. The composition of claim 1, wherein said at least one DNA fragment comprises an average of at least one methylated endonuclease recognition site of said targeted organism per 1.5 kb of said DNA fragment.

8. The composition of claim 1, wherein said at least one DNA fragment comprise an average of at least one endonuclease recognition site of an organism other than said targeted organism.

9. The composition of claim 1, wherein said at least one fragment comprise between 50 and 50,000 base pairs.

10. The composition of claim 1, wherein said carrier is an aqueous solution.

11. The composition of claim 10, said carrier further comprising at least one selected from the group consisting of divalent cations, monovalent cations, dimethyl sulfoxide, dithiothreitol, and hexamine cobalt and combinations thereof.

12. The composition of claim 11, wherein said cations are selected from the group consisting of Mg²⁺, Mn²⁺, Ca²⁺, Rb⁺, K⁺ and combinations thereof.

13. The composition of claim 1, wherein said targeted organism is selected from the group consisting of Salmonella enterica and said endonuclease recognition site is GATCAG; Salmonella enterica and said endonuclease recognition site is CAGAG; Salmonella enterica and said endonuclease recognition site is GAGNNNNNNRTAYG (SEQ ID NO: 1); Salmonella enterica and said endonuclease recognition site is GGTANNNNNRTGAA (SEQ ID NO: 2); Clostridium difficile and said endonuclease recognition site is CANNNNNNNTAAAG (SEQ ID NO: 3); Clostridium difficile and said endonuclease recognition site is AYGNNNNNNCTG (SEQ ID NO: 4); Clostridium difficile and said endonuclease recognition site is TAGNNNNNRTGAA (SEQ ID NO: 5); Staphylococcus aureus and said endonuclease recognition site is AGGNNNNNGAT (SEQ ID NO: 6); Staphylococcus aureus and said endonuclease recognition site is SCNGS; Streptococcus pneumoniae and said endonuclease recognition site is TCTAGA; wherein said targeted organism is Streptococcus pneumoniae and said endonuclease recognition site is GATC; Neisseria gonorrhoeae and said endonuclease recognition site is GGCC; Neisseria gonorrhoeae and said endonuclease recognition site is GGNNCC; Neisseria gonorrhoeae wherein said endonuclease recognition site is GGTGA; Neisseria gonorrhoeae and said endonuclease recognition site is GCCGGC; Neisseria gonorrhoeae and said endonuclease recognition site is RGCGCY; Neisseria gonorrhoeae and said endonuclease recognition site is CCACC; Neisseria gonorrhoeae and said endonuclease recognition site is CCGCGG; Neisseria gonorrhoeae and said endonuclease recognition site is GATC; Neisseria gonorrhoeae and said endonuclease recognition site is GCSGC; Neisseria gonorrhoeae and said endonuclease recognition site is GACNNNNNTGA (SEQ ID NO: 7); Neisseria gonorrhoeae and said endonuclease recognition site is GAGNNNNNTAC (SEQ ID NO: 8); Neisseria gonorrhoeae and said endonuclease recognition site is GCAGA; Campylobacter jejuni and said endonuclease recognition site is CATG; and Campylobacter jejuni and said endonuclease recognition site is GCANNNNNRTIA (SEQ ID NO: 9).

14. A method for the production of the composition of claim 1, comprising (i) providing a DNA fragment having at least 50% sequence homology to a sequence of said targeted organism; (ii) modifying at least one specific endonuclease recognition site of said synthesized DNA fragment to provide a modified DNA fragment comprising no more than 5 unprotected specific endonuclease recognition sites of said targeted organism; and (iii) isolating said modified DNA fragment.

15. The method of claim 14, wherein said modifying comprises chemically or enzymatically modifying.

16. The method of claim 14, wherein said modifying is selected from the group consisting of substitution, deletion and addition of at least one nucleotide.

17. (canceled)

18. The method of claim 14, wherein said modifying comprises methylating.

19-25. (canceled)

26. A method for the treatment of a condition caused by a pathogenic, parasitic or invasive microorganism or a symptom thereof, the method comprising administering to a subject in need thereof a therapeutically effective dose of the composition of claim 1.

27. The method of claim 26, wherein said condition is selected from the group consisting of Crohn's disease, a C. difficile infection, a urinary tract infection, a vaginal infection, a sexually transmitted disease, sepsis, inflammation and combinations thereof.

28-31. (canceled)


SEQUENCE LISTING (9 sequences)

SEQ ID NO: 1 - DNA, 14 bp, Artificial Sequence
  synthetic polynucleotide
  misc_feature (4)..(9): n is a, c, g, or t
  misc_feature (10)..(10): R =  Purine
  misc_feature (13)..(13): Y =  Pyrimidine
gagnnnnnnrtayg

SEQ ID NO: 2 - DNA, 14 bp, Artificial Sequence
  synthetic polynucleotide
  misc_feature (5)..(9): n is a, c, g, or t
  misc_feature (10)..(10): R =  Purine
ggtannnnnrtgaa

SEQ ID NO: 3 - DNA, 14 bp, Artificial Sequence
  synthetic polynucleotide
  misc_feature (3)..(9): n is a, c, g, or t
cannnnnnntaaag

SEQ ID NO: 4 - DNA, 12 bp, Artificial Sequence
  synthetic polynucleotide
  misc_feature (2)..(2): Y =  Pyrimidine
  misc_feature (4)..(9): n is a, c, g, or t
aygnnnnnnctg

SEQ ID NO: 5 - DNA, 13 bp, Artificial Sequence
  synthetic polynucleotide
  misc_feature (4)..(8): n is a, c, g, or t
  misc_feature (9)..(9): R =  Purine
tagnnnnnrtgaa

SEQ ID NO: 6 - DNA, 11 bp, Artificial Sequence
  synthetic polynucleotide
  misc_feature (4)..(8): n is a, c, g, or t
aggnnnnngat

SEQ ID NO: 7 - DNA, 11 bp, Artificial Sequence
  synthetic polynucleotide
  misc_feature (4)..(8): n is a, c, g, or t
gacnnnnntga

SEQ ID NO: 8 - DNA, 11 bp, Artificial Sequence
  synthetic polynucleotide
  misc_feature (4)..(8): n is a, c, g, or t
gagnnnnntac

SEQ ID NO: 9 - DNA, 12 bp, Artificial Sequence
  synthetic polynucleotide
  misc_feature (4)..(8): n is a, c, g, or t
gcannnnnrtta